(19) United States
(12) Patent Application Publication
(10) Pub. No.: US 2015/0359807 A1
(43) Pub. Date: Dec. 17, 2015

(54) Dietary Supplement Containing Vitamin A, D3 and Vitamin K2 and Uses Thereof

(71) Applicant: Supernutrition Life-Extension Research, Inc., Oakland, CA (US)

(72) Inventor: Michael Kevin MOONEY, W. Hollywood, CA (US)

(21) Appl. No.: 14/301,928

(22) Filed: Jun. 11, 2014

(51) Int. Cl. A61K 31/59; A61K 31/122; A61K 31/593; A61K 31/07; A23L 1/303; A61K 31/592

(52) U.S. Cl. CPC A61K 31/59; A23L 1/303; A61K 31/592; A61K 31/593; A61K 31/07; A61K 31/122; A23V 2002/00

(57) ABSTRACT

A composition comprising vitamin A, vitamin D and vitamin K₂ (K₂-MK-7) is provided. Advantageously, vitamin A is present in an amount of 1,000 IU to 10,000 IU, vitamin D is present in an amount of 400 IU to 10,000 IU and vitamin K₂ is present in an amount of 45 μg to 1,000 μg. In one advantageous form, vitamin A is retinol or an ester form thereof a method for limiting the accumulation of calcium and a method for limiting the occurrence of cardiovascular disease or events is provided by administering an effective amount of the present composition.

### FIELD OF THE INVENTION

The present invention relates to a composition comprising vitamin A, vitamin D and vitamin K₂ and uses thereof which include but are not limited to limiting the accumulation of calcium in a subject and limiting cardiovascular events such as strokes, heart failure and heart attacks.

### BACKGROUND OF THE INVENTION

The presence of calcium in arteries of mammals can lead to various afflictions. Although calcium accumulation is not a disease, in and of itself, arterial calcification has been proven to directly correlate to cardiovascular events such as stroke, heart failure and heart attacks. Since there is a strong correlation, medical professionals including physicians often recommend calcium screening to their at risk patients to quantify the extent of possible arterial calcification and thus generate a risk assessment. Using a calcium index (in which a calcium index is from 0 to 600), the higher the number, the more likely a person is to suffer from a cardiovascular event. Patients with a high calcium index numbers are generally put on rigorous diets, drugs and/or physical regimes to slow, inhibit or halt further calcium build-up in the arteries of these patients.

Unfortunately, dietary and exercise regimes often fail to produce a desired result in slowing, reducing or halting further calcification in the arteries of many subjects. Some reasons for the ineffectiveness include a low patient compliance, poor diet management and/or the inability of patients to find time and commit to a physical exercise schedule. In addition, conventional drugs used to treat calcium accumulation often have side effects that cause poor compliance or cessation of therapy. A reduction in calcium—even a reduction in the buildup of calcium from any given calcium-index is particularly fortunate because it coordinates to a significant reduction in mortality when measured against a control group.

In view of heart disease being a leading cause of death and financial disruption to families, society costs would be significantly affected from any means which slows or halts calcium buildup without side effects or discomfort of difficult dietary restrictions, unrealistic exercise requirements or drug side effects which may hinder compliance with arterial calcium abatement programs.

### SUMMARY OF THE INVENTION

The present invention, in various embodiments, is directed to a dietary supplement comprising vitamin A, vitamin D and vitamin K₂. Further, the present invention is directed to alleviating, slowing or halting calcium accumulation in a subject, including calcium buildup in the arteries of a subject. In addition, the present invention relates to alleviating complications which occur from the accumulation of calcium which includes but is not limited to strokes, heart failure and heart attacks.

The compositions in accordance with this disclosure include a formulation comprising a novel combination of three vitamins in advantageous and effective amounts (both in terms of the specific amounts of each constituent and the ratios or relative amounts of each constituent to the other two constituents) which, together, when present in the blood stream of a subject (e.g. an individual) produce a synergistic effect resulting in the ability to halt or slow calcium buildup in the subject's arteries. For example, delivering the present composition orally to the subject results in simultaneously introducing the constituents of the supplement into the blood stream of a subject at the same time and in the specific constituent amounts and ratio amounts to produce the desired effect.

Various compositions of this disclosure can be formulated as a tablet or as a chewable tablet or a melt tablet or dissolved in a liquid. If desired, the compositions can be in the form of an easy to swallow tablet. Doses include formulations of one to three tablets per day having specific amounts of vitamin A, vitamin D and vitamin K₂.

The present invention, in one form thereof, relates to a dietary supplement composition comprising vitamin A in an amount of 1000 IU to 10,000 IU, vitamin D in an amount of 400 IU to 10,000 IU and vitamin K₂ (K₂-MK-7, menaquinone-7) in an amount of 45 μg to 1,000 μg. The vitamin A can be retinol or an ester thereof selected from the group consisting of retinyl acetate and retinyl palmitate. The vitamin D can be vitamin D₃ (cholecalciferol) or vitamin D₂ (ergocalciferol). In various alternative further embodiments, the composition may comprise vitamin A in an amount of 1,250-5,000 IU, vitamin D in an amount of 1,000-4,000 IU and vitamin K₂ in an amount of 45-360 μg. Further, in one advantageous form, the dietary supplement may be in the form of a tablet, chewable form, melt form, or dissolved in a liquid wherein vitamin A is present in an amount of 1,666 IU, vitamin D is in an amount of 1,333 IU and vitamin K₂ is in an amount of 120 μg.

The present invention, in another form thereof, relates to a dietary supplement composition comprising a pharmaceutically acceptable carrier in combination with vitamin A, vitamin D and vitamin K₂.

The present invention, in another form thereof, relates to a method for limiting calcium accumulation in a subject comprising administering a composition comprising vitamin A in an amount of 1,000 IU to 10,000 IU, vitamin D in an amount of 400 IU to 10,000 IU and vitamin K₂ in an amount of 45 to 1,000 μg, to a subject in need of treatment therefrom, to thereby limit the accumulation of calcium. In one specific form, the treatment helps guide calcium away from soft body tissue and into bones and teeth.

The present invention in still yet another form thereof, relates to a method for limiting the occurrence of cardiovascular events and/or cardiovascular disease by administering a composition comprising vitamin A in an amount of 1,000 IU to 10,000 IU, vitamin D in an amount of 400 IU to 10,000 IU and vitamin K₂ in an amount of 45 to 1.000 μg to a subject in need of treatment therefrom to thereby limit the occurrence of cardiovascular events and/or cardiovascular disease.

### DETAILED DESCRIPTION

The present invention is directed to a composition comprising vitamin A, vitamin D and vitamin K₂. Further the present invention relates to specific formulations of compositions comprising vitamin A, vitamin D and vitamin K₂. In addition, the present invention relates to using compositions described in this disclosure for administration to a subject to limit the accumulation of calcium and to limit the occurrence of cardiovascular events such as strokes, heart failure and heart attacks.

The present advantageous compositions are the result of a synergistic effect which occurs between specific constituents of vitamin A, vitamin D and vitamin K₂, their specific amounts and the ratios of the specific amounts of each constituent relative to each other. Accordingly, it is a combination of the specific amounts of each of the constituents as well as their relative ratios to each other which produces the synergistic effect and results in the novel formulation and novel uses of the present compositions which includes limiting calcium accumulation and limiting cardiac events when administered to a subject.

The present compositions can be formulated to be suitable for children, adults, and over 50 i.e. the seniors market. Vitamin A can be present from 1,000 IU to 10,000 IU, vitamin D, such as vitamin D₃ from 400 IU to 10,000 IU and vitamin K₂ (menaquinone-7) also referred to as K₂-MK-7) in an amount of 45 μg to 1.000 μg. The combination of vitamin A, vitamin D and vitamin K₂ are nutrients which produce advantageous calcium levels in a subject and lead to drawing calcium away from soft tissue such as arteries and incorporating calcium into bones and teeth.

Vitamin A advantageously is in the form of vitamin A aldehyde also known as retinaldehyde and retinol having a chemical structure below

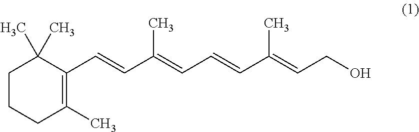 [SMILES: CC1=C(/C=C/C(C)=C/C=C/C(C)=C/CO)C(C)(C)CCC1]

Alternatively, retinol can be in one of its ester forms including retinyl acetate

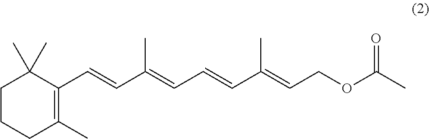 [SMILES: CC(=O)OC/C=C(C)/C=C/C=C(C)/C=C/C1=C(C)CCCC1(C)C]

and retinyl palmitate

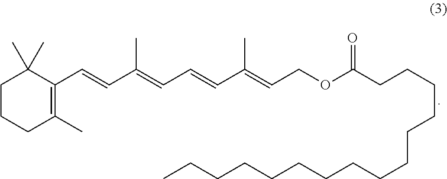 [SMILES: CCCCCCCCCCCCCCCC(=O)OC/C=C(C)/C=C/C=C(C)/C=C/C1=C(C)CCCC1(C)C]

Vitamin D can be in the form of vitamin D₂ (ergocaleiferol)

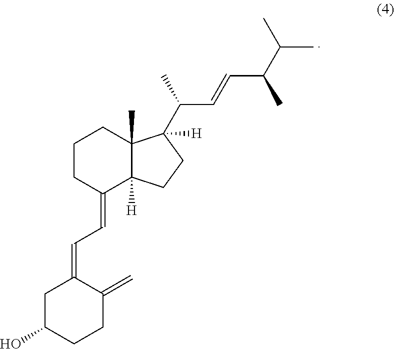 [SMILES: C=C1CC[C@H](O)C/C1=C/C=C1\CCC[C@]2(C)[C@@H]([C@H](C)/C=C/[C@H](C)C(C)C)CC[C@@H]12 ; "."]

Alternatively, vitamin D can be D₃, cholecalciferol also known as toxiferol

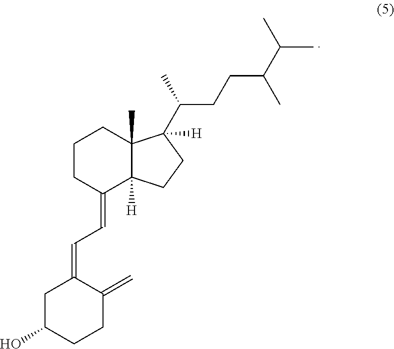 [SMILES: C=C1CC[C@H](O)C/C1=C/C=C1\CCC[C@]2(C)[C@@H]([C@H](C)CCC(C)C(C)C)CC[C@@H]12 ; "."]

The composition advantageously includes vitamin K₂

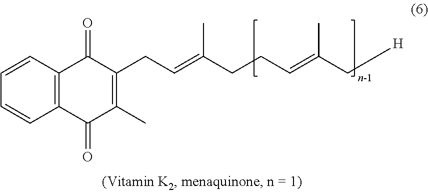

in the form of K₂-MK7

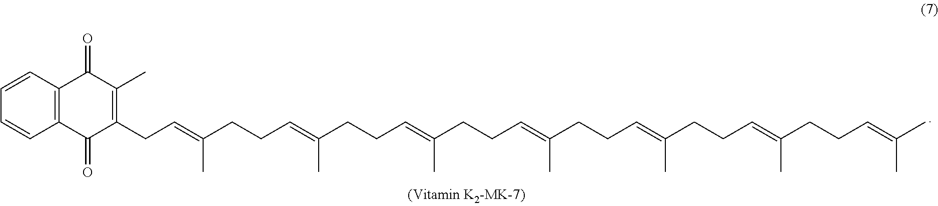 [SMILES: CC(C)=CCC/C(C)=C/CC/C(C)=C/CC/C(C)=C/CC/C(C)=C/CC/C(C)=C/CC/C(C)=C/CC1=C(C)C(=O)c2ccccc2C1=O ; "."]

The combination of constituents in the present compositions in their specific amounts or ranges, combined with ratios of the specific constituent amounts relative to each other, produces a unique synergistic effect. The synergistic effect is due to interactions between the constituents with each other and when present in, or administered to, a subject. Vitamin D stimulates chondrocytes and vascular smooth muscle tissue to produce matrix Gla-protein (MGP), which guides calcium away from soft tissues, such as arteries, that otherwise could become calcified, and kidneys, where calcium is a key factor in the development and progression of chronic kidney disease and nethrolithiasis (stone formation). However, MGP is only functional when activated (carboxylated) by vitamin K₂.

Vitamin D stimulates the production of the vitamin K₂-dependent proteins, osteocalcin and MGP, increasing vitamin D intake, even up to intake equaling 10,000 IU per day, which 21 studies have shown to be safe for long-term use for almost all adults, requires the minimum amount of vitamin K₂ (MK-7) to be 32.7 pig or higher, according to the Rotterdam study. In one advantageous formulation, doses of vitamin K₂ (K₂-MK-7) is from 45 μg to 180 μg or from 45 to 1,000 μg, to ensure optimal carboxylation of osteocalcin and MGP that cannot be satisfied by amounts of vitamin K₂ (MK-7) that are less than the Rotterdam study showed to be effective.

Vitamin A in the form of retinol or its ester retinyl acetate and retinyl palmitate and not beta carotene, moderates or slows the body's MGP production, functioning as a counterbalance to vitamin D's stimulation of MGP production. At the same time, it increases the rate at which bone is mineralized by its contribution to the production of osteocalcin. In this manner, adequate retinol helps to ensure that bone is created with sufficient density because of appropriate mineralization, as calcium is directed away from soft tissue by MGP and incorporated into bones and teeth through the action of carboxylated osteocalcin.

Vitamin A also improves vitamin D's stimulation of osteoblasts to produce osteocalcin to build bone, while also stimulating the proliferation of osteoclasts, which break bone down. This unique interaction between vitamins A and D yields optimal bone turnover resulting in bone regeneration. While collagen forms the main framework of the bone matrix, osteocalcin attracts and binds calcium within the bone matrix. However, like MGP, osteocalcin must be activated (carboxylated) by vitamin K₂ or it does not function optimally to bring calcium into bones and teeth. Thus, vitamins A, D and K₂ require each other for optimal calcium placement in the body, guiding calcium away from soft tissues, such as arteries, and into bones and teeth. In this manner, these three nutrients work together to support arterial health and bone and tooth health.

The present unique amounts of vitamin A, vitamin D and vitamin K₂ and their relative ratios to each other provide health benefits which are not found in prior compositions having different total amounts of constituents and different relative ratios of those constituents to each other.

While the terms used herein are believed to be well understood by one of ordinary skill in the art, definitions are set forth herein to facilitate explanation of the presently-disclosed subject matter.

Unless defined otherwise, all technical and scientific terms used herein have the same meaning as commonly understood by one of ordinary skill in the art to which the presently-disclosed subject matter belongs. Although any methods, devices, and materials similar or equivalent to those described herein can be used in the practice or testing of the presently-disclosed subject matter, representative methods, devices, and materials are now described.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±10.1% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

In some embodiments of the presently-disclosed subject matter, compositions, including pharmaceutical compositions comprise vitamin A, vitamin D (including D₃ also known as “cholecalciferol” and D₂ also known as “ergocalciferol”) and vitamin K₂ (including “menaquinone-7”, also known as K₂-MK-7). The compositions can be included with a pharmaceutically-acceptable vehicle, carrier, or excipient. In some embodiments, the composition is pharmaceutically-acceptable in humans. Also, as described further below, in some embodiments, the composition can be formulated as a therapeutic composition for delivery to a subject.

A pharmaceutical composition as described herein preferably comprises a composition that includes a pharmaceutical carrier such as aqueous and non-aqueous sterile injection solutions that can contain antioxidants, buffers, bacteriostats, bactericidal antibiotics and solutes that render the formulation isotonic with the bodily fluids of the intended recipient; and aqueous and non-aqueous sterile suspensions, which can include suspending agents and thickening agents. The pharmaceutical compositions used can take such forms as suspensions, solutions or emulsions in oily or aqueous vehicles, and can contain formulatory agents such as suspending, stabilizing and/or dispersing agents. Additionally, the formulations can be presented in unit-dose or multi-dose containers, for example sealed ampoules and vials, and can be stored in a frozen or freeze-dried or room temperature (lyophilized) condition requiring only the addition of sterile liquid carrier immediately prior to use.

In some embodiments, solid formulations of the compositions for oral administration can contain suitable carriers or excipients, such as corn starch, gelatin, lactose, acacia, sucrose, microcrystalline cellulose, kaolin, mannitol, dicalcium phosphate, calcium carbonate, sodium chloride, alginic acid calcium silicate or croscarmellose sodium. Disintegrators that can be used include, but are not limited to, microcrystalline cellulose, corn starch, sodium starch glycolate, and alginic acid. Tablet binders that can be used include acacia, methylcellulose, sodium carboxymethylcellulose, polyvinylpyrrolidone, hydroxypropyl methylcellulose, sucrose, starch, and ethylcellulose. Lubricants that can be used include magnesium stearates, stearic acid, silicone fluid, talc, waxes, oils, and colloidal silica. Further, the solid formulations can be uncoated or they can be coated by known techniques to delay disintegration and absorption in the gastrointestinal tract and thereby provide a sustained/extended action over a longer period of time. For example, glyceryl monostearate or glyceryl distearate can be employed to provide a sustained-/extended-release formulation. Numerous techniques for formulating sustained release preparations are known to those of ordinary skill in the art and can be used in accordance with the present invention, including the techniques described in the following references: U.S. Pat. Nos. 4,891,223; 6,004,582; 5,397,574; 5,419,917; 5,458,005; 5,458,887; 5,458,888; 5,472,708; 6,106,862; 6,103,263; 6,099,862; 6,099,859; 6,096,340; 6,077,541; 5,916,595; 5,837,379; 5,834,023; 5,885,616; 5,456,921; 5,603,956; 5,512,297; 5,399,362; 5,399,359; 5,399,358; 5,725,883; 5,773,025; 6,110,498; 5,952,004; 5,912,013; 5,897,876; 5,824,638; 5,464,633; 5,422,123; and 4,839,177; and WO 98/47491, each of which is incorporated herein by this reference.

Liquid preparations for oral administration can take the form of, for example, solutions, syrups or suspensions, or they can be presented as a dry product for constitution with water or other suitable vehicle before use. Such liquid preparations can be prepared by conventional techniques with pharmaceutically-acceptable additives such as suspending agents (e.g., sorbitol syrup, cellulose derivatives or hydrogenated edible fats); emulsifying agents (e.g. lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations can also contain buffer salts, flavoring, coloring and sweetening agents as appropriate. Preparations for oral administration can be suitably formulated to give controlled release of the active compound. For buccal administration, the compositions can take the form of capsules, tablets, melts or lozenges formulated in conventional manner.

The compositions can also be formulated as a preparation for implantation or injection. Thus, for example, the compositions can be formulated with suitable polymeric or hydrophobic materials (e.g., as an emulsion in an acceptable oil) or ion exchange resins, or as sparingly soluble derivatives (e.g., as a sparingly soluble salt).

Injectable formulations of the compositions can contain various carriers such as vegetable oils, dimethylacetamide, dimethylformamide, ethyl lactate, ethyl carbonate, isopropyl myristate, ethanol, polyols (glycerol, propylene glycol, liquid polyethylene glycol), and the like. For intravenous injections, water soluble versions of the compositions can be administered by the drip method, whereby a formulation including a pharmaceutical composition of the presently-disclosed subject matter and a physiologically-acceptable excipient is infused. Physiologically-acceptable excipients can include, for example, 5% dextrose, 0.9% saline, Ringer's solution or other suitable excipients. Intramuscular preparations, e.g., a sterile formulation of a suitable soluble salt form of the compounds, can be dissolved and administered in a pharmaceutical excipient such as Water-for-Injection, 0.9% saline, or 5% glucose solution. A suitable insoluble form of the composition can be prepared and administered as a suspension in an aqueous base or a pharmaceutically-acceptable oil base, such as an ester of a long chain fatty acid, (e.g., ethyl oleate).

For administration of a therapeutic composition as disclosed herein (e.g., a composition comprising vitamin A, vitamin D (including D₃ also known as “cholecalciferol” and D₂ also known as “ergocalciferol”) and vitamin K₂ (including “menaquinone-7”, also known as K₂-MK-7), conventional methods of extrapolating human dosage based on doses administered to a murine animal model can be carried out using the conversion factor for converting the mouse dosage to human dosage: Dose Human per kg=Dose Mouse per kg×12 (Freireich, et al., (1966) Cancer Chemother Rep. 50: 219-244). Doses can also be given in milligrams per square meter of body surface area because this method rather than body weight achieves a good correlation to certain metabolic and excretionary functions. Moreover, body surface area can be used as a common denominator for drug dosage in adults and children as well as in different animal species as described by Freireich, et al. (Freireich et al., (1966) Cancer Chemother Rep. 50:219-244). Briefly, to express a mg/kg dose in any given species as the equivalent mg/sq m dose, multiply the dose by the appropriate kg factor. In an adult human, 100 mg/kg is equivalent to 100 mg/kg×37 kg/sq m=3700 mg/m2.

Suitable methods for administering a therapeutic composition in accordance with the methods of the presently-disclosed subject matter include, but are not limited to, systemic administration, parenteral administration (including intravascular, intramuscular, and/or intraarterial administration), oral delivery, buccal delivery, subcutaneous administration, inter- and intramuscular, intraperitoneal administration, dermally (e.g., topical application), intratracheal installation, surgical implantation, transdermal delivery, local injection, intranasal delivery, and hyper-velocity injection/bombardment. Where applicable, continuous infusion can enhance drug accumulation at a target site (see, e.g., U.S. Pat. No. 6,180,082). In some embodiments of the therapeutic methods described herein, the therapeutic compositions are administered orally, intravenously, or intraperitoneally to thereby treat a disease, disorder, and/or condition as described herein below.

Regardless of the route of administration, the compositions of the presently-disclosed subject matter typically not only include an effective amount of a therapeutic agent, but are typically administered in amount effective to achieve the desired response. As such, the term “effective amount” is used herein to refer to an amount of the therapeutic composition sufficient to produce a measurable biological response e.g. reduction in the accumulation of calcium and/or the reduction of cardiac events. Actual dosage levels of active ingredients in a therapeutic composition of the presently-disclosed subject matter can be varied so as to administer an amount of the active compound(s) that is effective to achieve the desired therapeutic response for a particular subject and/or application. Of course, the effective amount in any particular case will depend upon a variety of factors including the activity of the therapeutic composition, formulation, the route of administration, combination with other drugs or treatments, severity of the condition being treated, and the physical condition and prior medical history of the subject being treated. Preferably, a minimal dose is administered, and the dose is escalated in the absence of dose-limiting toxicity to a minimally effective amount. Determination and adjustment of a therapeutically effective dose, as well as evaluation of when and how to make such adjustments, are known to those of ordinary skill in the art.

For additional guidance regarding formulation and dose, see U.S. Pat. Nos. 5,326,902; 5,234,933; PCT International Publication No. WO 93/25521; Berkow et al., (1997) The Merck Manual of Medical Information, Home ed. Merck Research Laboratories, Whitehouse Station, New Jersey; Goodman et al., (1996) Goodman & Gilman's the Pharmacological Basis of Therapeutics, 9th ed. McGraw-Hill Health Professions Division, New York; Ebadi, (1998) CRC Desk Reference of Clinical Pharmacology. CRC Press, Boca Raton, Fla.; Katzung, (2001) Basic & Clinical Pharmacology, 8th ed. Lange Medical Books/McGraw-Hill Medical Pub. Division, New York; Remington et al., (1975) Remington's Pharmaceutical Sciences, 15th ed. Mack Pub. Co., Easton, Pa.; and Speight et al., (1997) Avery's Drug Treatment: A Guide to the Properties, Choice, Therapeutic Use and Economic Value of Drugs in Disease Management, 4th ed. Adis International, Auckland/Philadelphia; Duch et al., (1998) Toxicol. Lett. 100-101:255-263.

As used herein, the terms “treatment” or “treating” relate to any treatment of a condition of interest (e.g. controlling calcium accumulation (e.g. in arteries) and cardiovascular disease), including but not limited to prophylactic treatment and therapeutic treatment. As such, the terms “treatment” or “treating” include, but are not limited to: preventing a condition of interest or the development of a condition of interest; inhibiting the progression of a condition of interest; arresting or preventing the further development of a condition of interest; reducing the severity of a condition of interest; ameliorating or relieving symptoms associated with a condition of interest; and causing a regression of a condition of interest or one or more of the symptoms associated with a condition of interest.

As used herein, the term “subject” includes human, animal and plant subjects. Thus, veterinary therapeutic uses are provided in accordance with the presently-disclosed subject matter. As such, the presently-disclosed subject matter provides for the treatment of mammals such as humans, as well as those mammals of importance due to being endangered, such as Siberian tigers; of economic importance, such as animals raised on farms for consumption by humans; and/or animals of social importance to humans, such as animals kept as pets or in zoos. Examples of such animals include but are not limited to: carnivores such as cats and dogs; swine, including pigs, hogs, and wild boars; ruminants and/or ungulates such as cattle, oxen, sheep, giraffes, deer, goats, bison, and camels; and horses. Also provided is the treatment of birds, including the treatment of those kinds of birds that are endangered and/or kept in zoos, as well as fowl, and more particularly domesticated fowl, i.e., poultry, such as turkeys, chickens, ducks, geese, guinea fowl, and the like, as they are also of economic importance to humans. Thus, also provided is the treatment of livestock, including, but not limited to, domesticated swine, ruminants, ungulates, horses (including race horses), poultry, and the like.

It will be appreciated that the function of the composition includes, but is not limited to adjusting vitamin levels in a subject, limiting calcium accumulation in arteries of the subject and limiting cardiovascular disease, etc., all as provided in this disclosure. In an advantageous form, the composition helps guide calcium away from body tissue and into bones and teeth. Accordingly, compositions, including pharmaceutical compositions consisting essentially of vitamin A, vitamin D, and vitamin K₂ have the aforementioned function(s), and related, requisite physical and chemical properties, to provide the aforementioned function(s) to adjust vitamin levels, limit calcium accumulation in arteries, and/or limit cardiovascular disease, etc., in a subject.

The practice of the presently-disclosed subject matter can employ, unless otherwise indicated, conventional techniques of cell biology, cell culture, molecular biology, transgenic biology, microbiology, recombinant DNA, and immunology, which are within the skill of the art. Such techniques are explained fully in the literature. See e.g., Molecular Cloning A Laboratory Manual (1989), 2nd Ed., ed. by Sambrook, Fritsch and Maniatis, eds., Cold Spring Harbor Laboratory Press, Chapters 16 and 17; U.S. Pat. No. 4,683,195; DNA Cloning, Volumes I and II, Glover, ed., 1985; Oligonucleotide Synthesis, M. J. Gait, ed., 1984; Nucleic Acid Hybridization, D. Hames & S. J. Higgins, eds., 1984; Transcription and Translation, B. D. Hames & S. J. Higgins, eds., 1984; Culture Of Animal Cells, R. I. Freshney, Alan R. Liss, Inc., 1987; Immobilized Cells And Enzymes, IRL Press, 1986; Perbal (1984), A Practical Guide To Molecular Cloning; See Methods In Enzymology (Academic Press, Inc., N.Y.); Gene Transfer Vectors For Mammalian Cells, J. H. Miller and M. P. Calos, eds., Cold Spring Harbor Laboratory, 1987; Methods In Enzymology, Vols. 154 and 155, Wu et al., eds., Academic Press Inc., N.Y.; Immunochemical Methods In Cell And Molecular Biology (Mayer and Walker, eds., Academic Press, London, 1987; Handbook Of Experimental Immunology, Volumes I-IV, D. M. Weir and C. C. Blackwell, eds., 1986.

### EXAMPLES

Following examples provide additional disclosure of specific preferable or advantageous formulations. However, the following examples are provided for exemplary purposes and are not the only formulations possible which produce an advantageous composition and provide the use for methods for limiting calcium accumulation and/or cardiovascular disease and events.

### Example 1

Example 1 is a dietary supplement composition which includes:

Vitamin A in an amount of 1,000 IU to 10,000 IU,

Vitamin D in an amount of 400 IU to 10,000 IU (10 μg to 250 μg), and Vitamin K₂ (K₂-MK-7) (menaquinone-7) in an amount of 45 μg to 1,000 μg.

### Example 1.1

The vitamin A is in the form of retinol. Accordingly, the amount of vitamin A will be between 300 μg and 3,000 μg.

### Example 1.2

The vitamin A is retinyl acetate (an ester form of retinol) and therefore the amount of retinyl acetate will be 344 μg to 3,440 μg.

### Example 1.3

The vitamin A is in the form of retinyl palmitate (another ester form of retinol) and therefore the amount of retinyl palmitate in the composition will be 550 μg to 5,500 μg.

### Example 2

Example 2 is a pharmaceutical composition in the form of a tablet which comprises vitamin A in an amount of 1,666 IU, vitamin D in an amount of 1,333 IU (33 μg) and vitamin K₂ (MK-7) in an amount of 120 μg.

### Example 2.1

Vitamin A is in the form of retinol (1,666 IU corresponding to 500 μg).

### Example 2.2

Vitamin A is in the form of retinol acetate (1,250 IU corresponding to 430 μg).

### Example 2.3

Vitamin A is in the form of retinyl palmitate (1,666 IU corresponding to 916.3 μg).

The formulations of Example 2, namely Examples 2.1-2.3, can be formulated in the form of a single tablet. A patient may be administered between 1 to 3 of these tablets per day, as needed based on a desired effect and the subject to be treated in accordance with this disclosure.

### Example 3

Example 3 is a composition comprising vitamin A in an amount of 5,000 IU, vitamin D₃ in an amount of 4,000 IU (75 μg) and vitamin K₂ (MK-7) in an amount of 360 μg.

### Example 3.1

Vitamin A is in the for Hi of retinol, 5,000 IU (1500 μg).

### Example 3.2

Vitamin A is in the form of retinyl acetate, 5,000 IU (1720 μg).

### Example 3.3

Vitamin A is in the form of retinyl palmitate, 5,000 IU (2700 μg).

It will now be clear that the present invention provides features and advantages not found in prior known compositions and treatments. Further, one of ordinary skill in the art will recognize that aspects of the present disclosure can be modified using routine techniques consistent with the present disclosure.

### REFERENCES CITED

Numerous references are cited in this disclosure including the following six (6) references. All references cited in this disclosure are hereby incorporated by reference.
1. Geleijnse J M, Vermeer C, Grobbee D E, et al. Dietary intake of menaquinone is associated with a reduced risk of coronary heart disease: the Rotterdam Study. J Nutr. 2004 November; 134(11):3100-5.
2. Adams J, Pepping J. Vitamin K in the treatment and prevention of osteoporosis and arterial calcification. Am J Health Syst Pharm. 2005 Aug. 1; 62(15):1574-81.
3. Farzanheh-Far et al. Transcriptional regulation of matrix Gla protein. Z Kardiol. 2001; 90(Suppl 3):38-42.
4. Oliva A et al. Effect of retinoic acid on osteocalcin gene expression in human osteoblasts. Biochem Biophys Res Commun. 1993 Mar. 31; 191(3):908-14.
5. Johansson S. Vitamin A and osteoporosis: experimental and clinical studies, Acta Universitatis Upsaliensis. Comprehensive Summaries of Uppsala Dissertations from the Faculty of Medicine, 1392 (2004):65.
6. Adams, K. Pepping, J. Vitamin K in the treatment and prevention of osteoporosis and arterial calcification. Am J Health-Syst Pharm. 2005; 62(15):1574-81.
7. Hathcock J N, Shao A, Vieth R, Heaney R. Risk assessment for vitamin D. Am J Clin Nutr. 2007 January; 85(1):6-18.

1. A dietary supplement composition comprising:
vitamin A in an amount of 1,000 IU to 10,000 IU,
vitamin D in an amount of 400 IU to 10,000 IU, and
vitamin K₂ (K₂-MK-7) (menaquinone-7) in an amount of 45 μg to 1,000 μg.

2. The dietary supplement composition of claim 1, wherein the vitamin A is retinol or an ester form thereof selected from the group consisting of retinyl acetate and retinyl palmitate.

3. The dietary supplement composition of claim 1, wherein the vitamin D is vitamin D₃ (cholecalciferol).

4. The dietary supplement composition of claim 1, wherein the vitamin D is vitamin D₂ (ergocalciferol).

5. The dietary supplement composition of claim 1, wherein vitamin A is present in an amount between 1,250-5,000 IU, vitamin D is present in an amount between 1,000-4,000 IU, and vitamin K₂ is present in an amount between 45-360 μg.

6. The dietary supplement composition of claim 1, wherein vitamin A is present in an amount of 1,666 IU, vitamin D is present in an amount of 1,333 IU, and vitamin K₂ is present in an amount of 120 μg.

7. The dietary supplement composition of claim 1, wherein vitamin A is present in an amount of 2,500 IU, vitamin D is present in an amount of 2,000 IU, and vitamin K₂ is present in an amount of 180 μg.

8. The dietary supplement composition of claim 1, wherein vitamin A is present in an amount of 3,750 IU, vitamin D is present in an amount of 3,000 IU, and vitamin K₂ is present in an amount of 135 μg.

9. The dietary supplement composition of claim 1, wherein vitamin A is present in an amount of 5,000 IU, vitamin D is present in an amount of 4,000 IU, and vitamin K₂ is present in an amount of 360 μg.

10. The dietary supplement composition of claim 1, further comprising a pharmaceutically acceptable carrier.

11. A dietary supplement composition in the form of a tablet comprising:
vitamin A in an amount of 1,000 IU to 10,000 IU,
vitamin D in an amount of 400 IU to 10,000 IU, and
vitamin K₂ (K₂-MK-7) (menaquinone-7) in an amount of 45 μg to 1,000 μg, and a pharmaceutically acceptable carrier.

12. The dietary supplement composition of claim 11, wherein vitamin A is present in an amount between 1,250-5,000 IU, vitamin D is present in an amount between 1,000-4,000 IU, and vitamin K₂ is present in an amount between 45-360 μg.

13. The dietary supplement composition of claim 12, wherein vitamin A is present in an amount of 1,250 IU, vitamin D is present in an amount of 1,000 IU, and vitamin K₂ is present in an amount of 45 μg.

14. The dietary supplement composition of claim 12, wherein vitamin A is present in an amount of 2,500 IU, vitamin D is present in an amount of 2,000 IU, and vitamin K₂ is present in an amount of 90 μg.

15. The dietary supplement composition of claim 12, wherein vitamin A is present in an amount of 3,750 IU, vitamin D is present in an amount of 3,000 IU, and vitamin K₂ is present in an amount of 135 μg.

16. The dietary supplement composition of claim 12, wherein vitamin A is present in an amount of 5,000 IU, vitamin D is present in an amount of 4,000 IU, and vitamin K₂ is present in an amount between 180-300 μg.

17. A method of limiting calcium accumulation in a subject comprising administering a composition comprising vitamin A in an amount of 1,000 IU to 10,000 IU, vitamin D in an amount of 400 IU to 10,000 IU, and vitamin K₂ (K₂-MK-7) (menaquinone-7) in an amount of 45 μg to 1,000 μg, to a subject in need of treatment therefrom, to thereby limit the accumulation of calcium.

18. The method of claim 17, wherein the treatment limits the accumulation of calcium in arteries of the subject.

19. A method of limiting the occurrence of cardiovascular events and/or cardiovascular disease by administering a composition comprising vitamin A in an amount of 1,000 IU to 10,000 IU, vitamin D in an amount of 400 IU to 10,000 IU, and vitamin K₂ (K₂-MK-7) (menaquinone-7) in an amount of 45 μg to 1,000 μg, to a subject in need of treatment therefrom, to thereby limit the occurrence of cardiovascular events and/or cardiovascular disease.

20. The method of claim 19, wherein the cardiovascular disease is selected from the group consisting of stroke, heart failure and heart attack.
